(19) United States
(12) Patent Application Publication
(10) Pub. No.: US 2020/0345055 A1
(43) Pub. Date: Nov. 5, 2020

(54) ACTIVATION OF CONDITIONALLY-EXPRESSED OLIGOSACCHARIDE PATHWAYS DURING FERMENTATION OF PROBIOTIC STRAINS

(71) Applicant: EVOLVE BIOSYSTEMS, INC., Davis, CA (US)

(72) Inventor: Steven FRESE, Oakland, CA (US)

(21) Appl. No.: 16/962,627

(22) PCT Filed: Jan. 18, 2019
(86) PCT No.: PCT/US2019/014097

§ 371 Date: Jul. 16, 2020

Related U.S. Application Data
(60) Provisional application No. 62/618,293, filed on Jan. 17, 2018.

(51) Int. Cl. A23L 33/00; A23C 9/12; A23C 9/20; A23D 9/007; A23K 10/18; A23K 20/163; A23L 29/30; A23L 33/135; C12N 1/20

(52) U.S. Cl. CPC A23L 33/40; A23C 9/12; A23C 9/203; A23D 9/007; C12N 1/20; A23K 20/163; A23L 29/30; A23L 33/135; A23K 10/18

(57) ABSTRACT

This invention relates generally to methods and compositions to achieve and maintain a desirable in vivo phenotype during fermentation and processing of food products for human or animal consumption. The methods and compositions of this invention require an activator that acts as a metabolic trigger for Mammalian Milk Oligosaccharide (MMO) consumption phenotype without necessarily requiring oligosaccharides (i.e a sugar polymer of 3 or more monosaccharides) within the fermentation medium. Compositions containing a non-oligosaccharide activator (e.g. monomers and dimers, and combinations thereof) may be used in fermentation processes of this invention. Turning on MMO-related genes without the use of oligosaccharides is one method of preparing activated commensal bacteria. Embodiments of this invention relate to a composition and a method for preparing a stable, activated and dormant form of commensal bacteria such as: bifidobacteria, lactobacilli or pediococci; for consumption by animals or humans, who are nursing or otherwise receiving MMOs as part of their diet.

### FIELD OF THE INVENTION

This invention relates generally to methods and compositions to achieve and maintain a desirable in vivo phenotype during fermentation and processing of food products for human or animal consumption. The methods and compositions of this invention require an activator that acts as a metabolic trigger for Mammalian Milk Oligosaccharide (MMO) consumption phenotype without necessarily requiring oligosaccharides (i.e a sugar polymer of 3 or more monosaccharides) within the fermentation medium. Compositions containing a non-oligosaccharide activator (e.g. monomers and dimers, and combinations thereof) may be used in fermentation processes of this invention. Turning on MMO-related genes without the use of oligosaccharides is one method of preparing activated commensal bacteria. Embodiments of this invention relate to a composition and a method for preparing a stable, activated and dormant form of commensal bacteria such as: bifidobacteria, lactobacilli or pediococci; for consumption by animals or humans, who are nursing or otherwise receiving MMOs as part of their diet.

### BACKGROUND

Human milk contains a significant quantity of complex oligosaccharides (HMO, which are up to 15% of total dry mass) in a form that is not usable as an energy source for the baby nor for most of the microorganisms in the gut of that baby. Certain microorganisms such as Bifidobacterium longum subsp. infantis (B. infantis) have the unique capability to consume specific complex oligosaccharides, such as those found in human or bovine milk (see U.S. Pat. Nos. 8,197,872 and 9,808,475, the contents of which are incorporated herein by reference). When B. infantis comes in contact with certain complex oligosaccharides (e.g., HMOs), a number of genes are specifically induced within the bacterium, and protein products of those genes act as enzymes and binding proteins which are responsible for the uptake and internal deconstruction of those complex oligosaccharides. The individual sugar components of those internalized oligosaccharides are then catabolized to provide energy for the growth and reproduction of that organism (Sela et al, 2008, PNAS, 105(48): 18964-69). It is generally understood that the genes required for HMO binding, transport and internalization in B. infantis are expressed in response to oligosaccharides in the environment (Garrido, 2011, PLoS ONE 6(3): e17315; Garrido, 2015, Scientific Reports, 5:1357).

B. longum subsp. longum and B. longum subsp. infantis diverged 5 million years ago. The genomes of these subspecies demonstrate adaptive differences which are reflected in their differential nutritional preferences (Locascio, et al., 2010, Applied and environmental microbiology, 76.22: 7373-7381).

All mammalian milk contains oligosaccharides (MMOs). Human milk has one of the highest concentrations and most diverse number of oligosaccharide structures compared to other mammals. MMOs of each mammalian species are sources of oligosaccharides for their own offspring. They may however also be fed to other mammals including humans to alter the MMO content of the diet which will promote the growth of bacteria containing MMO-consumption genes, such as infant-adapted Bifidobacterium. Bacteria not normally found in particular mammalian species may be selected for by their ability to partially use the MMO

The ability to bind and sequester intact HMOs inside the cell is an adaptive competitive strategy used by certain bifidobacteria to prevent monomers and other sugars being available as nutrients for other organisms. B. infantis is an example of an organism that internalizes the HMOs before breaking them down. In contrast, B. bifidum breaks HMOs down extracellularly. When HMOs are available in the environment, binding proteins, transport systems, and enzymes to break linkages within the oligosaccharides are transcriptionally induced to change protein expression profiles. These proteins are not normally present in an environment consisting of monomer sugars.

Activation of Bifidobacterium on milk oligosaccharides is described in WO/2016/065324, the contents of which are incorporated herein by reference. Activated organisms have increased adhesion to epithelial cells. Part of the activation phenotype is an increased expression of surface proteins that can interact with epithelial cells.

The ability to consume MMO is a hallmark of infant-adapted bacteria. Most preferably, a Bifidobacterium will have the ability to bind and transport intact MMO into the cell prior to breaking them down into constituent monomers that enter the bacterial metabolism. These genes are not normally turned on unless there are MMO present.

### SUMMARY OF INVENTION

The inventors discovered that certain monomers and/or dimers, alone or in combination, can replace milk oligosaccharides in a commercial fermentation media to activate MMO utilization pathways and to provide a nutrient carbon source. This was unexpected, as oligosaccharides containing 3-10 sugar residues are the only known substrates to activate this pathway, and neither monomers nor dimers have previously been known to induce expression of genes in this pathway.

This invention provides a method of preparing activated commensal bacteria selected from the group consisting of Bifidobacterium, Lactobacillus, and Pediococcus, and the method comprises culturing the commensal bacterial sp. in the presence of an activator selected from the compounds listed in Table 4, bacterial cells in the culture medium being activated by the presence of the activator included in the medium. In some embodiments, the activator compound from Table 4 is added in an amount sufficient to induce expression of a gene and/or a protein encoding for a surface binding protein, a sialidase, a fucosidase, or an alpha-N-acetylgalactosaminidase in the bacterial cells. Alternatively, the activator is added to the culture medium in sufficient amounts to increase enzymatic activity of a fucosidase, sialidase, or alpha-N-acetylgalactosaminidase. In some embodiments, the starting media composition comprises one or more compounds from Table 4 in an amount from 0.1 to 10%, preferably 3.0% by weight/vol of the media composition. In various embodiments, the activator constitutes a carbon source and consumption of the carbon source by commensal bacterial cells both increases cellular biomass and activates a transport system capable of internalizing one or more oligosaccharides having the structure of an oligosaccharide found in a mammalian milk before that oligosaccharide is hydrolyzed, these commensal bacterial cells being further capable of hydrolyzing the internalized oligosaccharide. Preferably, the mammalian milk is human, bovine, pig, rabbit, goat, sheep, camel, buffalo, or mixtures thereof. The activated commensal bacterial cells according to this invention generally have a higher binding affinity to mammalian mucosal cells than commensal bacterial cells of the same species cultivated on non-activating monomers or dimers.

Momomer and/or dimer activators are used according to this invention to activate Bifidobacterium and/or Lactobacillus and/or Pediococcus. The commensal bacteria may also come from mucin degrading bacteria such as Bacteroides and/or Akkermansia. The bacteria can be a single bacterial species of Bifidobacterium such as, but not limited to, B. adolescentis, B. animalis (e.g., B. animalis subsp. animalis or B. animalis subsp. lactis), B. bifidum, B. breve, B. catenulaturn, B. longum (e.g., B. longum subsp. infantis or B. longum subsp. longum), B. pseudocatanulatum, B. pseudolongum, a single bacterial species of Lactobacillus, such as, but not limited to, L. acidophilus, L. antri, L. brevis, L. casei, L. coleohominis, L. crispatus, L. curvatus, L. equi, L. fermentum, L. gasseri, L. johnsonii, L. mucosae, L. pentosus, L. plantarum, L. reuteri, L. rhamnosus, L. sakei, L. salivarius, L. paracasei, L. kisonensis., L. paralimentarius, L. perolens, L. apis, L. ghanensis, L. dextrinicus, L. shenzenensis, L. harbinensis, or a single bacterial species of Pediococcus, such as, but not limited to P. parvulus, P. lolii, P. acidilactici, P. argentinicus, P. claussenii, P. pentosaceus, or P. stilesii.

In preferred embodiments of this invention, activation of the commensal bacterial cells comprises upregulating Blon_0881 and Blon_2343 in B. infantis or the functional homologues in other bacterial species. In other embodiments, activation involves enhanced expression of glucosamine-6-phosphate isomerase and carbohydrate ABC transporter membrane protein from B. infantis or functional homologues from other Bifidobacterium, Lactobacillus, Pediococcus, Bacteroides, and Akkermansia. In still other embodiments, activation of the commensal bacterial cells comprises upregulating the genes selected from the group consisting of Blon_0042, Blon_R0015, Blon_R0017, Blon_R0021, Blon_R0022, Blon_2177, and combinations thereof, and/or downregulating genes selected from the group consisting of Blon_0518, Blon_0785, Blon_2167, Blon_2168 from B. infantis or the functional gene homologues from other Bifidobacterium, Lactobacillus, and Pediococcus and combinations thereof. In yet other embodiments, the commensal bacterial cells comprise an upregulated Blon_0042 gene from B. infantis or the functional gene homologues from other Bifidobacterium, Lactobacillus, and Pediococcus. In still other embodiments, the commensal bacterial cells comprise a downregulated Blon_2168 gene from B. infantis or the functional gene homologues from other Bifidobacterium, Lactobacillus and Pediococcus. In yet other embodiments, activation of the commensal bacterial cells comprise upregulating genes selected from the group consisting of Blon_0882, Blon_0881, Blon 0880, Blon_0879, Blon_2334, Blon_2335, Blon_2336, Blon_2337, Blon_2338, Blon_2339, Blon_2343, Blon_2344, Blon_2346, Blon_2347, and Blon_2331, or their functional homologues, that is, potentially related genes from a common ancestor, which may or may not differ in DNA sequence but encode a product with the same function in other species. In yet other embodiments, gene expression of one or more genes from Table 1, and/or protein expression or protein activity are monitored to determine activation. In some embodiments, genes from Table 2 are used to monitor activation. Preferably, the Bifidobacterium is B. longum, B. breve, B. bifidum, or B. pseudocatenulatum. More preferably, the Bifidobacterium is B. longum subsp. infantis, or the Bifidobacterium is B. breve or B. longum. Alternatively, the organism is from Pediococcus or Lactobacillus, Bacteroides or Akkermansia. In some embodiments, the activator is a monomer or dimer derived from chitosan and/or chitin.

In alternative embodiments, this invention provides a culture medium comprising an activator and activated Bifidobacterium. Preferably, the Bifidobacterium is B. longum, B. breve, B. bifidum, or B. pseudocatenulatum. More preferably, the Bifidobacterium is B. longum subsp. infantis, or the Bifidobacterium is B. breve. In various embodiments, the activator is present in culture media of this invention in an amount sufficient to induce a gene coding for a sialidase or a fucosidase in the Bifidobacterium. In some embodiments, the activator is present in the culture medium in an amount of from 0.1 to 10% by weight of the starting composition. Preferably 0.1 to 3.0% weight/volume of the starting composition will be the activator; more preferably 0.1 to 1%.

In particular embodiments, the activator is N-acetyl-D-glucosamine/galactosamine (NAG) which may be derived from chitosan and/or chitin; preferably, the culture medium in these embodiments further comprises chitin, chitosan, and/or fragments thereof or dimers which contain NAG or NAG derivatives. In particular embodiments, the activator is used is Lacto-N-Biose in Bifidobacterium, Lactobacillus, or Pediococcus specifc culture medium that meets minumium requirements for essential nutrients, cofactors and other compounds required to support the growth of the organism in the fermentor.

In some embodiments, the activated biomass is separated from the culture supernatant; the activator may be detected in the final product or removed through a washing step. In other embodiments, the activated supernatant is dried with the cells. In some embodiments, the cells are dried with MMO or other oligosaccharides. In these or other embodiments, excipients may be added to the recovered biomass, and the excipients may include an MMO or oligosaccharide. In some embodiments, the excipient contains lacto-N-biose, N-acetyllactosamine, fucosyllactose (FL) or derivatives of FL including but not limited to, lacto-N-fucopentose (LNFP) and lactodifucotetrose (LDFT); lacto-N-tetraose (LNT) and lacto-N-neotetraose (LNnT), and/or sialyl lactose or derivatives of sialyl lactose. The excipient may also be any low water activity powder such as lactose, or the dried cells are added to an oil suspension. This invention may also provide a composition comprising an activated Bifidobacterium and an activator from Table 4. In one preferred mode, the activator is N-acetylglucosamine or N-acetylgalactosamine (either may be referred to as NAG). More preferably, the NAG monomer is present in an amount of from 0.1 to 3.0% by weight of the composition, or the NAG monomer is present in an amount sufficient to induce expression of a gene coding for a sialidase or a fucosidase in the Bifidobacterium. The NAG may be derived from chitosan and/or chitin, and/or the composition further comprises chitosan and/or chitin fragments. In another preferred embodiment, the activator is LNB or N-acetlylactosamine. The Bifidobacterium may be B. longum, B. breve, B. bifidum, or B. pseudocatenulatum. Preferably, the Bifidobacterium is B. longum subsp. infantis, or the Bifidobacterium is B. breve. Preferably, the composition is a very low water activity composition. The composition may be in a dry form, optionally in a powdered form. In some embodiments, the composition is a powder with a water activity level of less than 0.35, less than 0.30, less than 0.25, less than 0.2, less than or less than 0.1. The composition may be spray-dried or freeze-dried; preferably, the composition is freeze-dried in the presence of a suitable cryoprotectant, where the cryoprotectant may be glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, or taurine, mammalian milk oligosaccharides, chitin, chitosan, other plant-based polysaccharides. Alternatively, the composition may be suspended in an oil, optionally, the oil is a medium chain triglyceride. In another embodiment, the composition is suspended in oligosaccharide syrup comprising at least 57% galactooligosacharides (GOS), where water activity is low enough to keep Bifidobacterium dormant. Any composition of this invention may be a food fed to an animal or a human. The food composition may include MMO or other oligosaccharides and an activated commensal bacterium as described herein.

### BRIEF DESCRIPTION OF THE FIGURES

FIG. 1. A comparison of fucosidase activity between a final product of B. infantis grown on N-acetylglucosamine (NAG) and a final product of B. infantis grown on glucose/lactose. B. infantis grown on 2.5% w/v NAG is compared to B. infantis grown on glucose/lactose. The absorbance due to the colorometric change resulting from fucosidase activity in the supernatant is measured using a spectrophotometer at 450 nm. The bar graph shows that NAG-activated cells had fucosidase activity whereas the cells grown on glucose/lactose did not.

FIG. 2. Average relative fold change in gene expression of the genes Blon_2343 and Blon_0881 in B. infantis coding a carbohydrate ABC transporter inner membrane protein and a glucosamine-6-phosphate isomerase or NagB, respectively. Different concentrations of N-acetylglucosamine (NAG) were added to the culture media used to ferment B. infantis. The cells were harvested after 6 hours (A) or 12 hours of growth (B) and RNA was isolated for quantitative reverse transcription PCR (qRT-PCR). Activation was assessed using specific primers for Blon_2343 and Blon_0881 and normalized to a housekeeping gene (Blon_0393). The dashed horizontal line represents no change in expression. All tested concentrations of NAG resulted in activation (2^{ΔΔCt}>1) of B. infantis. Cells grown in glucose alone (0 g/L NAG) show no activation.

FIG. 3. Average relative fold change in the expression of the genes coding for N-acetylglucosamine-6-phosphate deacetylase under different concentrations of N-acetylglucosamine (NAG) added to the culture media for Lactobacillus plantarum. Culture media with different concentrations of NAG (3, 12.5 and 20 g/L) were used to ferment Lactobacillus plantarum. The cells were harvested and RNA was isolated for qRT-PCR. Using specific primers for N-acetylglucosamine-6-phosphate deacetylase, activation was assessed using normalization to a housekeeping gene (rpoB) found in L. plantarum. Both 20 g/L and 12.5 g/L of NAG-activated L. plantarum but 3 g/L NAG did not.

### DETAILED DESCRIPTION OF INVENTION

An “activator” is defined in this invention as any monomeric or dimeric, or combinations of monomeric and/or dimeric carbohydrates capable of turning on one or more of the genes in Table 1 related to HMO binding, transport or degradation or one or more of the genes in Table 2 related to NAG consumption. Examples of activators are listed in Table 4. This invention can use, but is not limited to those activators listed in Table 4; activators according to this invention do not include oligosaccharides.

“Activation” is defined as a change in gene expression for genes involved in consumption of MMOs, such as HMO or structurally related glycans, over the expression level in those same strains growth on lactose or glucose in a fermentation process. Genes involved in HMO function are defined as genes from the 5 HMO clusters defined in Sela 2008 and Locascio 2010, whether or not they are actualy found in those clusters. The genes that may be activated according to this invention include those listed in Table 1 and Table 2. Homologues of these genes in other organisms may also be expressed by activation according to this invention. Activation may be determined as an increase in gene expression of specified genes and/or functional readouts of the encoded proteins such as sialidase or fucosidase or an alpha-N-acetylgalactosaminidase enzyme activity. Activation is measurable in the fermentation media, harvested bacterial cells, and/or in the bulk dried concentrate and/or final product mixed with an excipient to dilute the product to the final concentration.

An activator is able to induce expression of genes in an organism but does not necessarily promote the selective growth of the organism over other organisms during an in vivo or an in vitro competition assay.

An “activated cell” is defined as one that has an increased expression in at least one of the genes found in the HMO clusters in B. infantis, or functionally homologous genes in other bacterial species. The activated cell may show increased expression of solute binding proteins (SBP), extracellular enzymes, or ABC transporters on the cell surface or the change may be intracellular.

A “commensal gut bacterium” is a member of the gut microbiome that is not known to cause disease.

“Functional homologues” are potentially related genes from a common ancestor, which may or may not differ in DNA sequence but encode a product with the same function in related species.

A “non-oligosaccharide” is defined as a carbohydrate having 1 or 2 hexoses or pentoses—e.g. 1-2 degrees of polymerization.

An “oligosaccharide” is defined as a carbohydrate having 3-20 sugar residues or degrees of polymerization from any source.

A “mammalian milk oligosaccharide (MMO)” is defined as an oligosaccharide from mammalian milk, whether it is purified or enriched or detectable in a dairy product, as long as the oligosaccharide is not subject to metabolism by digestive enzymes expressed in the mammalian genome. MMO includes individual synthetic structures oligosaccharide equivalent to those present in a mammalian milk including milk from human, bovine, equine, porcine, goat, camel, water buffalo, and sheep. An oligosaccharide regardless of its source (plant or animal) that functionally behaves as an MMO and can be mimicked by the monomer, dimer, or upstream or downstream metabolic intermediate is covered by this invention.

A “carbon source” is defined as a component of the medium that is a carbon-containing molecule able to promote the growth of the organism i.e. increase biomass. It may or may not also possess a feature of activating the genes in Table 1 that relate to HMO binding, transport or degradation.

A “primary carbon source” is defined as a component of the medium that when present will drive the increase in biomass and yield of the fermentation product. Used in this context it does not have the capacity to activate the cells.

The “total carbon source” in the medium will provide material to support the exponential growth of the organism, or doubling time, to produce a sufficient yield of activated product. The carbohydrates driving both rapid growth and activation may not come from the same molecules, but they can. The total carbohydrate/total carbon source for the type of fermentations covered by this invention will typically be in the range of 1-3% weight/volume or 10-30 g/L, but can be lower or higher. Residual sugars may be detectable in the spent media. A primary carbon source is one used to drive the yield; while the activator may be a primary carbon source, its function is to change the gene expression. A primary carbon source plus an activator can equal the total carbohydrate or the total carbon source.

Description of the Activation Phenotype

In some embodiments, the activation phenotype involves upregulating one or more of the genes contained in one or more HMO gene clusters. Examples of these gene clusters from B. infantis are listed in Table 1. In other species functional homologues will have different prefixes and numbers. The function of the respective gene is the important part relative to this invention.

TABLE 1
List of genes that are associated with HMO consumption in B. infantis as described
in Locascio 2010. The prefix Blon refers to genes in B. infantis.
B. infantis specific gene clusters

| H1 | H2 | H3 | H4 | H5 | Urease |
|---|---|---|---|---|---|
| HMO cluster | Fucosidase | Fucosidase | Sialic Acid | Lacto-N-biose | Cluster |
| Blon_2331 | Blon_0243 | Blon_0423 | Blon_0641 | Blon_2171 | Blon_0104 |
| Blon_2332 | Blon_0244 | Blon_0424 | Blon_0642 | Blon_2172 | Blon_0105 |
| Blon_2334 | Blon_0245 | Blon_0425 | Blon_0643 | Blon_2173 | Blon_0106 |
| Blon_2336 | Blon_0246 | Blon_0426 | Blon_0644 | Blon_2174 | Blon_0107 |
| Blon_2342 | Blon_0247 | | Blon_0645 | Blon_2175 | Blon_0108 |
| Blon_2343 | Blon_0248 | | Blon_0646 | Blon_2176 | Blon_0109 |
| Blon_2344 | | | Blon_0647 | Blon_2177 | Blon_0110 |
| Blon_2347 | | | Blon_0648 | | Blon_0111 |
| Blon_2348 | | | Blon_0649 | | Blon_0112 |
| Blon_2350 | | | Blon_0650 | | Blon_0113 |
| Blon_2351 | | | Blon_0651 | | Blon_0114 |
| Blon_2352 | | | | | Blon_0115 |
| Blon_2354 | | | | | |
| Blon_2355 | | | | | |
| Blon_2357 | | | | | |
| Blon_2359 | | | | | |
| Blon_2360 | | | | | |
| Blon_2361 | | | | | |

The activation phenotype can relate to the capture, internalization and/or metabolism of the HMO; and/or relate to the binding affinity for epithelial cells; as well as catabolism of milk sugar monomers, dimers, or oligosaccharides; and/or production of tryptophan or indole derivatives. The activation phenotype can involve any inducible pathway that accompanies preparation of a stable, activated bacterial phenotype with an improved ability to consume fiber/oligosaccharides within the colon of an animal or human¹, where the oligosaccharides include but are not limited to oligosaccharides and/or other fiber, but may more particularly refer to MMO. This invention provides for production of the activation phenotype prior to consumption and/or use of oligosaccharides/fiber in said animal or human. In some cases, it is for a newborn infant of the said animal or human. ¹ The activated cells will also demonstrate an improved ability to consume oligos in vitro.

Activation may specifically include genes related to NAG consumption: Blon_0882 (N-acetylglucosamine 6-phosphate deacetylase (EC 3.5.1.25)), Blon_0881 (glucosamine-6-phosphate isomerase), Blon_0880 (NagC/XylR-type transciptional regulator), Blon_0879 (Sugar kinase of the NBD/HSP70 family) from B. infantis. (Table 2). Functional homologues of any of these genes from other species can be used to measure activation in their respective species.

TABLE 2
B. Longum/B. infantis genes associated with NAG
pathways that may represent activation. Functional
homologues in other bacterial species may be used.

| Gene | Function |
|---|---|
| Blon_0882 | N-acetylglucosamine 6-phosphate |
| | deacetylase (EC 3.5.1.25) |
| Blon_0881 | glucosamine-6-phosphate isomerase, |
| Blon_0880 | NagC/XylR-type transciptional regulator |
| Blon_0879 | Sugar kinase of the NBD/HSP70 family |

In various embodiments, activation of Bifidobacterium infantis cells requires upregulation of Blon_0881 (glucosamine-6-phosphate isomerase) and Blon_2343 (carbohydrate ABC transporter membrane protein) and/or homologues of glucosamine-6-phosphate isomerase and carbohydrate ABC transporter membrane protein from other Bifidobacterium, Lactobacillus and Pediococcus (Table 3), and activation can be confirmed by monitoring expression of these genes. In other embodiments, an additional gene is selected from one of the clusters listed in Table 1 or Table 2, or its functional homologues in other species. In some embodiments, activation is alternatively measured using one or more genes that are not Blon-0881 or Blon_2343 or its species-specific functional homologues. In some embodiments, activation of the HMO phenotype involves upregulation of a transcriptional regulator, such as Blon_0042 from B. infantis. In other embodiments, activation genes may be selected from one or more of Blon_R0015, Blon_R0017, Blon_R0021, Blon_R0022, transfer RNA (tRNA) of the amino acids valine, leucine, phenylalanine, and aspartate, respectively. In further embodiments activation may involve monitoring downregulation of Blon_0518, Blon_0785 (ABC-type nitrate/sulfonate/bicarbonate transport system, periplasmic component), Blon_2167 (hypothetical protein), Blon_2168 (phage shock protein C (PspC) family protein) alone or in addition to one or more genes that are also activated by the activation source selected from Table 4.

Table 1 and Table 2 describe the gene loci in B. infantis and the gene functions whose homologues can be found in other species that can reliably turn on part or all of the HMO phenotype (activation phenotype) and are the basis for describing activation in either Bifidobacterium, Lactobacillus and/or Pediococcus. In some embodiments, a Blon_2343 gene is selected from HMO cluster 1 and another gene marker of activation from a region outside one of the HMO clusters. Typically, a gene is also selected that is constitutively expressed within the organism to normalize the data. Table 3 shows a suitable set of genes for monitoring activation. Functional gene equivalents or homologues can be found for Lactobacillus and Pediococus.

TABLE 3
List of Locus Tags for genes to determine activation
using a constitutive reference gene from B. infantis.
The reference gene normalizes the data.

| Locus Tag | Gene product | Gene Purpose |
|---|---|---|
| Blon_0881 | glucosamine-6-phosphate isomerase or | Activation |
| | NAG B | |
| Blon_2343 | carbohydrate ABC transporter inner | Activation |
| | membrane protein | |
| Blon_0393 | cysteinyl-tRNA synthetase | Reference |

In other embodiments, other microorganisms such as non-infant Bifidobacterium, Lactobacillus and Pediococcus, Bacteroides, Akkermansia are activated using these methods specifically to turn on the genes associated with NAG or other carbon utilization pathways which may represent a part of the HMO phenotype turned on in B. infantis. In another mode of this invention the NAG pathway in Lactobacillus is activated using sugars unrelated to the NAG pathway, such as LNB or N-acetly-lactosamine. The rpo gene in Lactobacillus may be used as a reference or housekeeping gene.

Description of Activation

In some embodiments, activation is considered to be when the relative expression of the HMO cluster genes is upregulated compared to the same genes when the cell is grown on lactose or glucose. The genes selected for activation are known to be upregulated when grown in the presence of MMO compared to lactose or glucose, so if their expression is increased relative to the reference gene, it is sufficient to describe activation. In some embodiments, gene activation relates to Blon_0881 and Blon_2343 and their expression is greater than 1; that is, when the delta delta cycle threshold (2^{-ΔΔ}^{_{Ct}}) is greater than 1, 2^{-ΔΔ}^{_{Ct}} is calculated using Blon_0393, or another constitutively expressed gene as a reference. The result (activation) is the fold change (2^{-ΔΔ}^{_{Ct}}>1) of gene expression, determined based on the 2^{-ΔΔ}^{_{Ct}} method. (Livak K and Schmittgen T. (2001). Analysis of relative gene expression data using qRT-PCR and the 2^{-ΔΔ}^{_{Ct}} Method. Methods: 25: 402-8). In other embodiments, other genes from Table 1 are used in addition to Blon_0881 and Blon_2343.

In other embodiments, a functional readout of activation is measured using sialidase and/or fucosidase and/or an alpha-N-acetylgalactosaminidase activity. In other embodiments, activation is determined by both gene activation and a functional readout. In some embodiments activation is monitored, using solute binding proteins or ABC transporters. In some embodiments, cell binding is monitored for activation.

Description of the Different Monomers and Dimers that can be Used to Activate Cells

A selected group of monomers or dimers that includes, but is not limited to N-acetyl glucosamine/galactosamine (NAG), dimers containing at least one moiety of NAG, fucose, and/or sialic acid, lacto-N-Biose, galacto-N-biose, hexose disaccharide (eg. Fuc α1,2Galβ) may be used in fermentation processes according to this invention as activators of the MMO pathway(s), and optionally as a primary carbon source. These activators are listed in Table 4.

TABLE 4
List of sources that can be used in this invention to
act as activators alone or in combination to activate
conditionally expressed oligosaccharide pathways
Source

N-acetyl-glucosamine or N-acetyl-galactosamine = NAG
Dimeric N-acetylglucosamine, dimeric N-acetylgalactosamine
Fucose
Sialic Acid
Lacto-N-biose = galactose-N-acetylglucosamine
N-acetyl-lactosamine
Galacto-N-Biose = galactose-N-acetylgalactosamine
Fuc α1,2Galβ

NAG is a sugar that is a monomeric residue component of MMO. It is released by beta-hexosaminidase (Sela et al, 2008, PNAS, 105(48): p 18964-69) from oligosaccharides containing these sugars. Garrido, et al., 2012 (Anaerobe, 18: 430-435) described the release and utilization of NAG from HMO by B. infantis. It was concluded that NAG was used for peptidoglycan synthesis rather than glycolysis in B. infantis. NAG is an important nutrient for the establishment and maintenance of the intestinal epithelium. Infants use free NAG monomer present in the lumen of the gut. NAG monomer may be used by the infant and/or other bacteria in the small intestine and not reach the large intestine where it could be used directly by B. infantis. NAG has also been suggested as a sweetening agent in food and beverages and as a food additive for beneficial effects on the body (US 2007/0259094).

Chitin and Chitosan are polysaccharides containing repeating N-acetyl glucosamine and are a source of monomers. There is some evidence for a human chitinase in the stomach. Bovine cartilage, shark cartilage, fungi all contain chitin. Commercially, Chitin and Chitosan are currently derived from algae or insect larvae. The polysaccharides when broken down by chitinase and/or by chemical/physical methods into monomers and dimers are useful for this invention.

Sialic acid and fucose are monomers that are part of the composition of some mammalian oligosaccharides and plant derived oligosaccharides. These monomer sugars attached as residues in the oligosaccharide structure make it difficult for bacteria to access this carbon source. A limited number of bacteria possess the ability to break the bond between these sugars, and therefore the rest of the oligosaccharide structure is protected from being broken down. On the other hand, free sialic acid and fucose are readily used by the host and by pathogens [see Wang, B., Annual Review of Nutrition, 2009, Vol. 29:177-222; Ng et al., Nature. 2013. 502, 96-99],

Lacto-N-biose (LNB) and N-acetyllactosamine are core dimers that are part of human milk oligosaccharide. Lacto-N-biose has been produced using a one pot enzymatic reaction to be used as a bifidus factor for growth in vivo (Biosci. Biotechnol. Biochem, 71 (8):2101-2104, 2007). Alternatively galacto-N-biose may be used.

αFuc 1,2Galβ is a core moiety of a number of N-linked fucosylated glycans of the mucus layer on the intestinal epithelium surface. Only a limited number of intestinal bacterial species, such as Bifidobacterium encode the complete repertoire of enzymes necessary to metabolize mucins as energy source, thus possessing an adaptive advantage over other bacteria to colonize and survive in the intestine. (Front. Genet. 6: 81, 2015)

Description of Fermentation Processes

In some embodiments, one or more of the sources listed in Table 4 can be used as the total carbon source required in the fermentation to increase the biomass of Bifidobacterium and activate the cells before they are harvested from a fermenter. In other embodiments, the sources listed in Table 4 are added as a percentage of the total carbohydrate added to the fermentation, e.g. 5%, 10%, 20%, 30%, 40%, 50%, 60%, 70%, 80% or 90%, while the remainder of the carbon source comes from glucose, galactose or lactose to make 100% of the carbon source at the beginning of the fermentation. In other embodiments, an activating compound (carbon source) from Table 4 may be added to a fermentation during the late exponential phase to turn on the oligosaccharide pathway. In other embodiments the carbon sources listed in Table 4 are fed (supplied) to the fermenter intermittently or continuously via one or more feed streams during cultivation. In other embodiments, cells are re-suspended in a solution containing the sources in Table 4. In other embodiments, the cells are transferred to a secondary fermentation vessel containing the sources listed in Table 4.

In some embodiments, a composition of fermentation media is prepared that contains an activator as the sole carbon source (100% of the total carbohydrate present in the fermentation media). A fermentation typically will start with the carbon source (carbohydrate) at 1-3% of the final composition (weight/volume). In these embodiments, the activator may increase biomass or yield and turn on the right genes for HMO consumption before the cells are harvested.

In other embodiments, a composition of fermentation media is prepared that contains one or more simple fermentable sugars, such as glucose, galactose or lactose as the primary carbon source and an activator (i.e NAG, N-acetyllactosamine, fucose, sialic acid or another compound listed in Table 4) are both added at the start of fermentation. The primary carbon source may be at least 90%, 85%, 80%, 75%, 70%, 65%, 60%, 55%, 50%, 45%, 40%, 35%. 30%, 25%, 20%, 15%, 10% of the total carbon source. The remainder of the carbon source may be the activator at 10%, 15%, 20%, 25%, 30%, 35%, 40%, 45%, 50%, 55%, 60%, 65%, 70%, 75%, 80%, 85%, 90% of the total carbohydrate present (carbon source) in the starting media.

In other embodiments, a composition that contains a simple fermentable sugar, such as glucose, galactose or lactose as the primary carbon source is used to initiate the fermentation and contains up to 50%, 60%, 70%, 80%, 90% or 100% of the required carbon for the size of fermentation being conducted. In the same embodiment, the activator is added during the late exponential phase when a simple fermentable sugar is reduced from the starting levels. The activator (i.e NAG or any of the other sugars listed in Table 4) is added in late exponential phase to be 5%, 10%, 15%, 20%, 25%, 30%, 35%, 40%, 45%, 50%, 55%, 60%, 65%, 70%, 75%, 80, 85%, 90%, 95%, 100% of the amount of total carbohydrate present at the start of the fermentation. The activating compound from Table 4 may be left over in the spent media and dried with the organism or it may be completely used before the cells are harvested.

A method of preparing/priming/activating a bacteria such as Bifidobacterium, Lactobacillus, or Pediococcus to stimulate production of enzymes that are necessary to consume HMO prior to being put into a dormant state. Any of the compositions described herein may be prepared by cultivating a bifidobacteria in an axenic culture (e.g., a culture with genetic homogeneity), and the culture will become “activated” if one or more of the compounds listed in Table 4 are included in the medium. In various embodiments, any of the compositions described herein can be made by isolating bifidobacteria; purifying the bacteria; inoculating a fermenter with the purified strains of the bifidobacteria; culturing the bifidobacteria in the presence of one or more activators from Table 4; and harvesting the cells. Fermentations for bifidobacteria may be carried out in stirred tank fermenters of commercial volume (e.g., 1-500 m³) which are maintained under anaerobic conditions throughout the fermentation process. The fermentation can include the steps of providing at least one or more activators from Table 4 at any time during the course of the fermentation in a liquid culture at a level of at least 1 g/L, typically from about 1-50 g/L, or 2-20 g/L, or 5-10 g/L as a sole, or supplementary, carbon source to activate the cells.

A method of preparing a two-stage fermentation to separate the yield generating steps from activation steps. Here, the first exponential growth phase is driven by a simple carbon source like glucose to stationary phase. The spent media is removed and new media is added to the fermentation tank containing an activator from Table 4. The second phase is driven by the activator to turn the genes on. In this second stage the cells may or may not come out of a lag phase. A return to exponential growth is one sign the genes have been activated, but is not necessary to demonstrate activation.

In various embodiments, sialidase and/or fucosidase gene expression or enzyme assay activity are one means of confirming activation in a culture or a freeze-dried powder. Increased expression of solute binding proteins are also a functional consequence of activating the HMO pathway. (This increases Bifidobacterium binding to epithelial cells.) Blon0042 is a transcriptional regulator gene for the HMO cluster and its functional analogs in other strains. Proteins that are at least 70% homologous to Blon0042 may be detected as an indicator of activation.

Other organisms such as Lactobacillus or Pediococcus, or mucin-degrading bacteria such as Bacteroides and Akkermansia have part of the HMO phenotype that may relate to NAG utilization, and as such, this method can be used to activate them for growth on other structures.

Low water activity is required to keep organisms dormant during long-term storage. In some embodiments, the stability of the activated Bifidobacterium or other bacteria in powder form requires water activity less than 0.35, less than 0.25, less than 0.2, less than 0.1. In other embodiments, anhydrous oils are used to maintain the organism in a stable dormant state in an oil suspension including, but not limited to, medium chain triglyceride (MCT), a natural food oil, an algal oil, a fungal oil, a fish oil, a mineral oil, a silicon oil, a phospholipid, and/or a glycolipid. The oils may be used alone or in combination. Oils have low water activity, and edible oils, e.g. medium chain triglycerides, mineral oils, vegetable oils, can be blended with the activated Bifidobacterium, or Pediococcus, or Lactobacillus, or Bacteroides or Akkermansia. Syrups or other excipients with low enough water activity with or without other stabilizers may be used to keep cells dormant until use.

The composition can also include a food source that contains all the nutritional requirements to support the life of a healthy mammal. That mammal may be, but is not limited to, an infant, an adolescent, an adult, or a geriatric adult. The food source can be a nutritional formulation designed for a human, buffalo, camel, cat, cow, dog, goat, guinea pigs, hamster, horse, pig, rabbit, sheep, monkey, mouse, or rat. For example, the food source can be a food source for an infant human which further comprises a protein such as, but not limited to, a milk protein, a cereal protein, a seed protein, or a tuber protein. The food source can be mammalian milk including, but not limited to, milk from human, bovine, equine, caprine, or porcine sources. The food can also be a medical food or enteral food designed to meet the nutritional requirements for a mammal, for example, a human.

The composition may also comprise from about 5 to 90% of dietary glycans from a mammalian source including, but not limited to a human, swine, or bovine species.

The activated bacteria may be in single use or multiple use packaging in a vial, sachet, stickpack, capsule, tablet or other food product.

### EXAMPLES

### Example 1. A Fermentation to Produce Dried Activated Bifidobacterium infantis

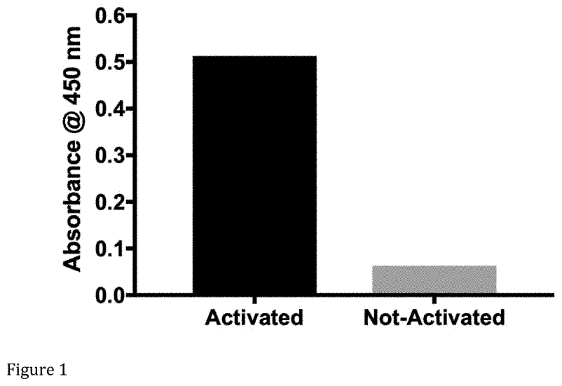

A fermentation media was made that contained NAG as 100% of the carbon source. This represented 2% of the total media composition (wt/vol). The media also contained sources of nitrogen, minerals, reducing agents and was autoclaved prior to addition of a B. infantis inoculum. The fermentation was carried out under anaerobic conditions for up to 72 hours, until the fermentation reached the stationary phase. The cells were separated from the spent media and concentrated. A cryoprotectant was mixed with the cells to stabilize before freeze-drying. Once dried, the formulation was analyzed for fucosidase activity compared to a dried formulation that had been grown on glucose/lactose. The activated B. infantis release nitrophenol from a colorless aryl-substituted glycoside via fucosidases expressed by activated B. infantis. In comparison, the assay fails to demonstrate released nitrophenol (yellow color) when incubated with control cells of B. infantis grown on glucose/lactose (data not shown). The colorimetric difference was confirmed using a spectrophotometer (FIG. 1).

### Example 2. Activation of B. infantis

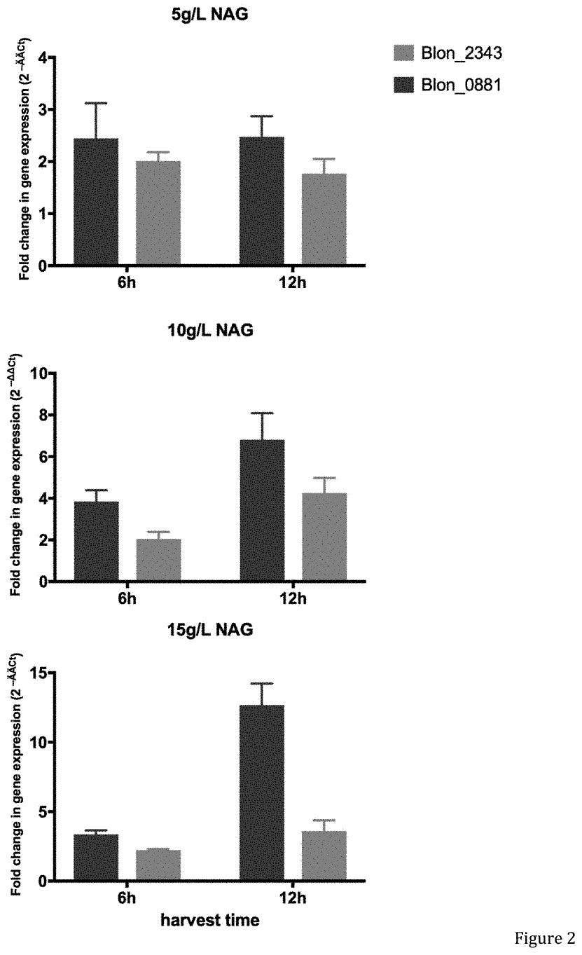

A series of fermentations were conducted where B. infantis was grown on various concentrations of NAG and glucose shown in Table 5. The cells were grown for a determined period of time (i.e 6 and 12 hours) and spun down. The supernatant was removed and the cells lysed. RNA was extracted. The cells were tested for increase in gene expression of Blon_0881 and Blon_2343 by qRT-PCR relative to Blon_0393. Results are in FIG. 2.

TABLE 5
NAG/Glucose Proportions

| NAG g/L | Glucose g/L |
|---|---|
| 20 | 0 |
| 15 | 5 |
| 10 | 10 |
| 5 | 15 |
| 0 | 20 |

### Example 3. Activation and Drying of a B. infantis Biomass

Cells grown on lactose are harvested and re-suspended in a buffered media containing 50% glucose and 50% sialic acid, representing 1% of the total media composition (wt/vol) for 2 hours. The cells are tested for increase in gene expression of Blon_0881 and Blon_2343 by qRT-PCR relative to Blon_0393. The cells are separated from the activation media and concentrated. A cryoprotectant that includes MMO as one component is mixed with the cells to stabilize before freeze-drying. Once dried the formulation is mixed with an excipient containing lactose and fucosyllactose.

### Example 4. B. infantis EVC001 Activation Using N-Acetyllactosamine

B. infantis EVC001 (Bifidobacterium longum subsp. infantis EVC001 deposited under ATCC Accession No. PTA-125180) was grown under 3 different experimental conditions for 16 hours. The carbon source was added to the initial culture media in the following amounts: 1) 20 g/L glucose; 2) 10 g/L N-acetyl-lactosamine and 10 g/L glucose; and 3) 10 g/L NAG and 10 g/L glucose. The cells were harvested and RNA was extracted to look specifically at gene expression of Blon_0881 and Blon_2343 by qRT-PCR relative to Blon_0393 and expressed as 2^{-ΔΔ}^{_{Ct}}. Results are shown in the table 6 below. Activation is determined by comparing against the control (glucose). A result greater than 1 is considered activated N-acetyl-lactosamine is an activator of B. infantis EVC001.

TABLE 6

| | Mean 2^{-ΔΔCt} | Mean 2^{-ΔΔCt} | |
|---|---|---|---|
| Experimental condition | BLON_0881 | BLON_2343 | Interpretation |
| 20 g/L glucose | 1 | 1 | Not activated |
| 10 g/L N-acetyl-lactosamine + 10 g/L | 10.50 | 6.94 | Activated |
| glucose | | | |
| 10 g/L NAG + 10 g/L glucose | 3.41 | 2.10 | Activated |

### Example 5. Production of an Activated Lactobacillus plantarum Fermentate

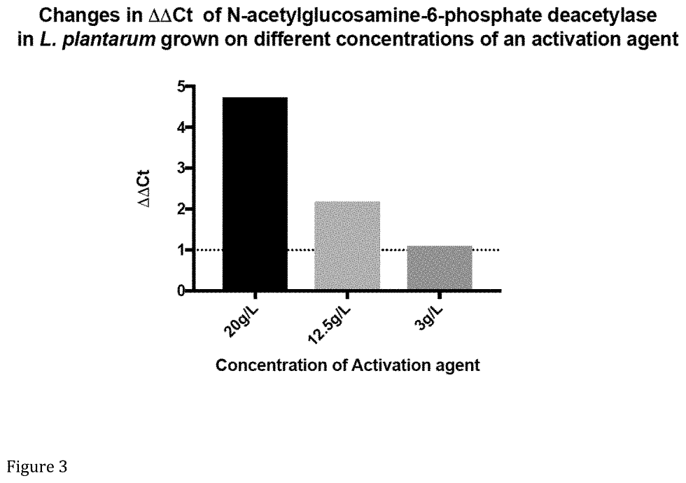

Three different fermentation media compositions were made that contained: 1) 100% NAG (25 g/L); 2) 50% NAG (12.5 g/L) with 50% Lactose (12.5 g/L); and 3) 12.5% NAG (3 g/L) with 87.5% lactose (22 g/L). This represented about 2% of the total media composition (wt/vol). The media also contained sources of nitrogen, minerals, reducing agents and was autoclaved prior to addition of L. plantarum inoculum. The fermentation was carried out under anaerobic conditions for 24 hours until the cells reached the stationary phase. The cells were harvested and RNA extracted (Sambrook, Joseph. & Russell, David W. & Cold Spring Harbor Laboratory. (2012). Molecular cloning: a laboratory manual. Cold Spring Harbor, N.Y: Cold Spring Harbor Laboratory). Specific primers for Lactobacillus were used for the following gene N-acetylglucosamine-6-phosphate deacetylase (activation gene) using normalization to a housekeeping gene (rpoB) found in L. plantarum. Results showed that activation only occurred when NAG represented 50% or 100% of the carbon source. The results are presented in FIG. 3.

### Example 6. Activation of Pediococcus During Fermentation

A fermentation media is made that contains 100% lactose. This represents 1% of the total media composition (wt/vol). The media also contains sources of nitrogen, minerals, reducing agents and is autoclaved prior to addition of Pediococcus spp. inoculum. The fermentation is carried out under anaerobic conditions for 12 hours. A concentrated solution of NAG (25% wt/vol) is supplied to the fermentation vessel via a feeding stream to reach a final concentration of 1% (wt/vol) in the total media composition. The fermentation continues for 4 more hours. The cells are separated from the spent media and concentrated. A cryoprotectant that includes chitin as one component is mixed with the cells to stabilize before freeze-drying. Once dried the formulation is mixed with an excipient containing lactose and NAG. Cells are checked for alpha-N-acetylgalactosaminidase at the gene and/or functional level.

1. A method of preparing activated commensal bacteria, said method comprising culturing said commensal bacterial sp. in the presence of an activator,
wherein said commensal bacteria are selected from the group consisting of Bifidobacterium, Lactobacillus, Pediococcus, Bacteroides, and Akkermansia,
wherein said activator is a carbohydrate monomer or dimer, and
wherein bacterial cells in the culture medium are activated by the presence of the activator included in the medium.

2. The method of claim 1, wherein the activator is present at the initiation of the culture.

3. The method of claim 1, wherein the activator is added after lag phase of the culture.

4. The method of claim 3, wherein the activator is added no earlier than the middle of the exponential phase, but at least by the end of the exponential phase.

5. The method of any one of claims 1 to 4 wherein activated commensal bacteria are recovered from the culture.

6. The method of claim 5, wherein the recovered bacteria are spray-dried or freeze-dried.

7. A culture medium suitable for use in the method of any one of claims 1 to 6,
wherein said culture medium is adapted for culturing commensal bacteria,
wherein the fermentation ingredients of the culture medium are food grade and/or suitable for human or animal consumption, and
wherein said activator is a carbohydrate monomer or dimer.

8. The culture medium of claim 7, wherein the culture medium is anaerobic and further wherein the culture medium comprises commensal bacteria selected from the group consisting of Bifidobacterium, Lactobacillus, Pediococcus, Bacteroides, and Akkermansia, which are activated.

9. The culture medium of claim 7 or claim 8, wherein the culture medium also comprises a non-activating carbon source.

10. The culture medium of claim 9, wherein the activator constitutes 100%-1% of the total carbon source and non-activating carbohydrate constitutes 0%-99% of the total carbon source.

11. A composition comprising an activator and activated commensal bacteria recovered from culture medium according to any one of the preceding claims.

12. The composition of claim 11, wherein the composition further comprises chitosan and/or chitin fragments, and/or mammalian milk oligosaccharides.

13. The composition of claim 12 wherein the composition further comprises one or more of Lacto-N-biose, N-acetyllactosamine, Lacto-N-tetraose or Lacto-N-neo-tetraose

14. The composition of any one of claims 11-13, wherein the composition is a powder with a water activity level of less than 0.35, less than 0.30, less than 0.25, less than 0.2, less than or less than 0.1.

15. The composition of any one of claims 11-13, wherein the composition is an anhydrous composition.

16. The composition of any one of claims 11-15, wherein the composition is in a powdered form.

17. The composition of any one of claims 11-15, wherein the composition is in a dry form.

18. The composition of any one of claims 11-17, wherein the composition is spray-dried or freeze-dried.

19. The composition of any one of claims 11-18, wherein the composition is dried in the presence of a suitable cryoprotectant.

20. The composition of claim 19, wherein the cryoprotectant is glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, or taurine, mammalian milk oligosaccharides, chitin, chitosan, other polysaccharides.

21. The composition of any one of claims 11-20, wherein the composition is suspended in an oil.

22. The composition of claim 21, wherein the oil is a medium chain triglyceride.

23. The composition of any one of claims 11-20, wherein the composition is suspended in oligosaccharide syrup at GOS at least 57% where water activity is low enough to keep Bifidobacterium dormant.

24. The composition of any one of claims 11-23, wherein the composition is a food suitable to be fed to an animal or a human.

25. The composition of any one of claims 11-24 wherein the composition is formulated to be fed to a newborn animal, especially a newborn human.

26. The method, the culture medium, or the composition of any one of the preceding claims, wherein said activator is selected from the compounds listed in Table 4,

27. The method, the culture medium, or the composition of any one of the preceding claims, wherein the activator is selected from N-acetyl-glucosamine/galactosamine (NAG), dimeric N-acetyl-glucosamine, dimeric N-acetyl-galactosamine, fucose, sialic acid, lacto-N-biose, N-acetyl-lactosamine, galacto-N-bios, or Fuc-α-1,2-Gal-β.

28. The method, the culture medium, or the composition of claim 26, wherein the activator is selected from N-acetyl-glucosamine/galactosamine (NAG), or dimeric N-acetyl-glucosamine.

29. The method, the culture medium, or the composition of claim 26, wherein the activator is selected from Lacto-N biose or N-acetyl-lactosamine

30. The method, the culture medium, or the composition of any one of the preceding claims, wherein once activated, commensal bacterial cells express a transport system capable of internalizing one or more oligosaccharides before said oligosaccharide is hydrolyzed and consequently said commensal bacterial cells are further capable of hydrolyzing said internalized oligosaccharide, wherein said oligosaccharide has the structure of an oligosaccharide found in a mammalian milk.

31. The method of claim 30, wherein the mammalian milk is human, bovine, pig, rabbit, goat, sheep, camel, buffalo milk, or mixtures thereof.

32. The method of any one of the preceding claims, wherein the activated commensal bacterial cells have a higher binding affinity to mammalian mucosal cells than commensal bacterial cells of the same species cultivated on non-activating monomers or dimers.

33. The method, the culture medium, or the composition of any one of the preceding claims, wherein the activator is added in an amount from 0.1% to 10% of the culture medium (w/v), preferably from 0.1% to 3%.

34. The method, the culture medium, or the composition of any one of the preceding claims, wherein the activator is present in an amount sufficient to induce expression of a gene encoding for a sialidase, a fucosidase, or an alpha-N-acetylgalactosaminidase, or genes listed in Table 1 or Table 2 in the bacterial cells.

35. The method, the culture medium, or the composition of any one of the preceding claims, wherein activation of the commensal bacterial cells comprises upregulating Blon_0881 and Blon_2343 in B. infantis or the functional homologues in other bacterial species, said homologues being expressed during activation of said other bacterial species.

36. The method, the culture medium, or the composition of any one of the preceding claims, wherein activation is glucosamine-6-phosphate isomerase and carbohydrate ABC transporter membrane protein from B. infantis or functional homologues from other Bifidobacterium, Lactobacillus and Pediococcus.

37. The method, the culture medium, or the composition of any one of the preceding claims, wherein activation of the commensal bacterial cells comprises upregulating the genes selected from the group consisting of Blon_0042, Blon_R0015, Blon_R0017, Blon_R0021, Blon_R0022, Blon_2177 and combinations thereof, and/or downregulating genes selected from the group consisting of Blon_0518, Blon_0785, Blon_2167, Blon_2168 from B. infantis or the functional gene homologues from other Bifidobacterium, Lactobacillus and Pediococcus and combinations thereof.

38. The method, the culture medium, or the composition of any one of the preceding claims, wherein the commensal bacterial cells comprise an upregulated Blon_0042 gene from B. infantis or the functional gene homologues from other Bifidobacterium, Lactobacillus, Pediococcus, Bacteroides, or Akkermansia.

39. The method, the culture medium, or the composition of any one of the preceding claims, wherein the commensal bacterial cells comprise a downregulated Blon_2168 and/or Blon_2177 gene from B. infantis or the functional gene homologues from other Bifidobacterium, Lactobacillus and Pediococcus.

40. The method, the culture medium, or the composition of any one of the preceding claims, wherein activation of the commensal bacterial cells comprise upregulating genes selected from the group consisting of Blon_0882, Blon_0881, Blon 0880, Blon_0879, Blon_2334, Blon_2335, Blon_2336, Blon_2337, Blon_2338, Blon_2339, Blon_2344, Blon_2346, Blon_2347, and Blon_2331 or their functional homologues in other species.

41. The method, or the culture medium, or the composition of any one of the preceding claims, wherein the commensal bacteria are Bifidobacterium and the bacterium may be B. longum (subsp. longum, or infantis) B. B. breve, B. bifidum or B. pseudocatenulatum.

42. The method, or the culture medium, or the composition of claim 41 wherein the Bifidobacterium longum is B. longum subsp. infantis.

43. The method, or the culture medium, or the composition of claim 41, wherein the Bifidobacterium is B. breve.

44. The method, or the culture medium, or the composition of any one of the preceding claims, wherein the commensal bacteria are Lactobacillus and the Lactobacillus may be L. rhamnosus, L. reuteri, or L. plantarum.

45. The method, or the culture medium, or the composition of claim 44, wherein the Lactobacillus is L. plantarum.

46. The method, or the culture medium, or the composition of any one of the preceding claims, wherein the commensal bacteria are Pediococcus and the bacterium may be P. acidilactici or P. pentosaceus.
